(19) United States
(12) Patent Application Publication
(10) Pub. No.: US 2024/0120089 A1
(43) Pub. Date: Apr. 11, 2024

(54) SYSTEM AND METHOD FOR PROVIDING A DYNAMIC MEDICAL PRACTICE QUEUING PLATFORM

(71) Applicant: The MyTurnPlus Stock Corporation, a California Corporation, CLOVIS, CA (US)

(72) Inventors: Steffen Michael Meeks, Clovis, CA (US); Erick Joseph Green, Jr., Clovis, CA (US); HAIDER KHAN, Lahore (PK); ALI RAZA, Bahawalpur (PK)

(21) Appl. No.: 18/309,428

(22) Filed: Apr. 28, 2023

Related U.S. Application Data
(60) Provisional application No. 63/413,332, filed on Oct. 5, 2022.

(51) Int. Cl. G16H 40/67; G16H 10/60; G16H 40/20

(52) U.S. Cl. CPC G16H 40/67; G16H 10/60; G16H 40/20

(57) ABSTRACT

Systems and methods for providing a dynamic medical practice queueing platform via at least one patient user mobile device in operable connection with a network. An application server is in operable communication with the network to host an application program for displaying, via a display module, a virtual patient queue. The application program includes a user interface module for providing access to the virtual patient queue through the display module. A geofencing module determines the location of a user in reference to a geofenced location. A virtual patient queue module determines a time of arrival of a patient using the current location of the patient and positions the patient within a virtual queue.

### CROSS-REFERENCE TO RELATED APPLICATION

This application claims priority under 35 U.S.C § 119(e) to U.S. Provisional Patent Application No. 63/413,332 filed on Oct. 5, 2022 and titled “SYSTEM AND METHOD FOR PROVIDING A DYNAMIC MEDICAL PRACTICE QUEUING PLATFORM”, the entire contents of which are hereby incorporated by reference.

### FIELD

The embodiments generally relate to computerized systems and methods for providing a dynamic medical practice queuing platform based on a facility's patient flow and real-time real-world conditions including patient location, traffic, and others.

### BACKGROUND

Medical care facilities are often inundated with patients, especially those operating as urgent care (UC) facilities. Commonly, urgent care clinics have unpredictable patient flows and manage patients experiencing a wide range of symptoms and severity of injuries and illnesses. In many clinics, static waitlists, requiring an administrator to update the waitlist by hand, result in patients being treated in the order they arrive (i.e. first in first out) unless an exceptionally serious patient emergency exists. As can be expected, this archaic manual system results in long wait times for many patients.

The most common method of queuing patients at UC facilities is according to static slotted appointments. Static slotted appointments are generally appointment blocks or timeslots of pre-defined length (e.g., fifteen or thirty minutes) in which patients are scheduled to be seen by healthcare providers. Under the static slotted appointment method, all appointments are estimated to require a similar amount of time. Of course, each patient's needs are unique, and the static slotted appointment method is rigid and generally inflexible. Fixed timeslots fail to account for individual patient needs and thereby lead to inefficiencies in providing care. When patients require less time than the timeslots allow, time is wasted. When patients require more time than the timeslots allow, appointments may be rushed.

Due to the fact that UC clinics have unpredictable patient flows, with walk-ins experiencing, for example, fractures, lacerations, and serious nose bleeds, needs exist at UC clinics that would provide queueing software with dynamic updates based on current, real-time patient volume and flows. Such systems could also beneficially dynamically communicate estimated time for patient visits based on current actual patient quantities measured in a queue.

### SUMMARY

This summary is provided to introduce a variety of concepts in a simplified form that is disclosed further in the detailed description of the embodiments. This summary is not intended to identify key or essential inventive concepts of the claimed subject matter, nor is it intended for determining the scope of the claimed subject matter.

Typical static appointment slot software seen in ambulatory patient engagement solutions is nearly worthless in the hyper dynamic queuing ecosystem of existing urgent care facilities.

The embodiments described herein remedy such situations and relate to a system for providing a dynamic medical practice queueing platform at least one user computing device in operable connection with a network. An application server is in operable communication with the network to host an application program for displaying, via a display module, a virtual patient waitlist. The application program includes a user interface module for providing access to virtual patient waitlist through the display module. A geofencing module determines the location of a user in reference to a geofenced location. A virtual patient waitlist module determines a time of arrival of a patient using the current location of the patient and to position the patient within a virtual waitlist queue.

According to various embodiments, the systems and methods disclosed herein determine patient volume and patient flows at medical facilities, such as urgent care facilities, to dynamically communicate the estimated time for patient visits based on actual volumes of patients in a UC facility's queue, patient location, and commute time, coupled to additional algorithms. These systems and method are implemented in some embodiments as smart phone applications, designed to provide UC customers/patients with a dynamic queuing ecosystem. They set forth algorithms for the management of a virtual waiting room and are capable of exponentially reducing UC wait room times in some instances. Such algorithms operate on a set of data parameters processed at the back end of wireless network connected applications run on smart mobile devices.

In operation, the various embodiments provide for patient users to be placed in a virtual queue in a virtual waiting room upon the selection of desired urgent care facility that the user selects from a list of local UC facilities displayed in the application running on their mobile device. Such virtual queues are maintained by algorithms run as software process that determine estimated time for patient users to be seen at the urgent care facility based on the user/customers current location (e.g. as determined according to their mobile device) and is updated in real-time based on geo-fencing techniques as well as travel time and mapping systems.

Back-office personal computers (PCs) located in the UC can be updated periodically (e.g. monthly) with data input including one or more of the percent of customers that received treatment and the mandatory sit-down time associated with the type of treatment provided in order to teach the system the treatment patterns by region. In embodiments where treatments are injections or shots, such data can drive the virtual waiting room exit data which may influence the geofence data (e.g. by modifying the distance and/or expected time when a patient user needs to leave or should arrive at the UC facility). The development of an injection data feature over time gives the systems and methods a dynamic algorithm by which machine learning and adjustments can become ever more precise and finely tuned to changing real-world conditions.

In some aspects, patient users receive real-time notifications from the system and/or UC facility predicting optimal departure and receives map-based travel directions (e.g. driving, walking, bicycling, or public transportation directions) from a user's current location to the selected UC facility.

In some aspects, patient users/customers are prompted by the application to capture images of their health insurance card or other documents via a mobile device camera. These images and/or other files can be stored on their mobile device and/or uploaded via a network to one or more system servers.

In some aspects, the application embodies machine learning algorithms that enhance the efficiency and accuracy of the application. The UC facility data can be periodically collected (e.g. on a monthly basis) to train the machine learning models and contribute to continuous improvement in application's accuracy.

In some aspects, greater flexibility is provided by the embodiments herein. Healthcare providers are able to tailor the length of appointments based on patient needs and healthcare provider availability. Rather than strict schedule adherence, real-time adjustments can be made with dynamic queuing and time can be adjusted to better accommodate individual patient needs.

In some aspects, improved patient experience is provided by the embodiments described herein. Since dynamic appointment queuing can eliminate wait times and allow for more personalized care, patient satisfaction with UC facilities overall can be vastly improved. This can also reduce the number of walkouts, which may be potential patients who grow tired of waiting for their appointment and simply leave a UC facility without treatment or paying.

In some aspects, dynamic appointments according to the embodiments herein allow for more patients to be seen effectively and this better use of time can lead to increased revenue for UC facilities.

### BRIEF DESCRIPTION OF THE DRAWINGS

Aspects, features, and advantages of specific embodiments of the present disclosure will become more apparent from the following description with reference to the accompanying drawings:

FIG. 1 illustrates a block diagram of a computing system, according to some embodiments;

FIG. 2 illustrates a block diagram of a computing system and an application program, according to some embodiments;

FIGS. 3A-3B illustrate a block diagram of an application flow which the system may provide and a user may interact with while utilizing the system, according to an example embodiment;

FIG. 4A shows a splash screen of a mobile device user interface, according to an example embodiment;

FIG. 4B shows a location permissions screen of a mobile device user interface, according to an example embodiment;

FIG. 4C shows a location permissions follow-up screen of a mobile device user interface, according to an example embodiment;

FIG. 4D shows a notification permissions follow-up screen of a mobile device user interface, according to an example embodiment;

FIG. 4E shows a login screen of a mobile device user interface, according to an example embodiment;

FIG. 4F shows an account creation screen of a mobile device user interface, according to an example embodiment;

FIG. 4G shows verify contact number screen of a mobile device user interface, according to an example embodiment;

FIG. 4H shows an on-boarding page display screen of a mobile device user interface, according to an example embodiment;

FIG. 5 shows an urgent care facility listing screen of a mobile device user interface, according to an example embodiment;

FIG. 6A shows an urgent care facility listing screen of a mobile device user interface with expanded information, according to an example embodiment;

FIG. 6B shows a travel mode selection screen of a mobile device user interface with expanded information, according to an example embodiment;

FIG. 6C shows a successful appointment creation screen of a mobile device user interface with expanded information, according to an example embodiment;

FIG. 7 shows an urgent care facility queue screen of a mobile device user interface, according to an example embodiment;

FIG. 8 shows an updated urgent care facility queue screen of a mobile device user interface, according to an example embodiment;

FIG. 9 shows a map screen of a mobile device user interface, according to an example embodiment;

FIG. 10A shows a user profile editing screen of a mobile device user interface, according to an example embodiment;

FIG. 10B shows a family member profile editing screen of a mobile device user interface, according to an example embodiment;

FIG. 10C shows an insurance upload screen of a mobile device user interface, according to an example embodiment;

FIG. 11 shows a camera screen of a mobile device user interface, according to an example embodiment;

FIG. 12A shows an image selection screen of a mobile device user interface, according to an example embodiment;

FIG. 12B shows a thank you screen of a mobile device user interface, according to an example embodiment;

FIG. 13 shows a block diagram of an administrative flow, according to an example embodiment;

FIG. 14A shows an urgent care facility queue information administrator user interface screen, according to an example embodiment;

FIG. 14B shows an urgent care facility queue information administrator user interface screen, according to an example embodiment;

FIG. 14C shows an urgent care facility queue information administrator user interface screen, according to an example embodiment;

FIG. 15 shows an urgent care facility report administrator user interface screen, according to an example embodiment; and

FIG. 16 shows an urgent care facility notification administrator user interface screen, according to an example embodiment; and

FIG. 17 shows system integration diagram, according to an example embodiment.

### DETAILED DESCRIPTION

The specific details of the single embodiment or variety of embodiments described herein are to the described system and methods of use. Any specific details of the embodiments are used for demonstration purposes only, and no unnecessary limitations or inferences are to be understood thereon.

Before describing in detail exemplary embodiments, it is noted that the embodiments reside primarily in combinations of components and procedures related to the system. Accordingly, the system components have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein.

In this disclosure, the various embodiments may be a system, method, and/or computer program product at any possible technical detail level of integration. A computer program product can include, among other things, a computer-readable storage medium having computer-readable program instructions thereon for causing a processor to carry out aspects of the present disclosure.

In general, the embodiments provided herein relate to a system for providing a dynamic medical practice queuing platform designed to provide urgent care (UC) facilities and patients with a means for dynamically queuing patients in a virtual waiting room. The system allows administrative users to manage the virtual waiting room. Further, patients utilize the system to enter the waiting room queue and reduce time spent in the waiting room. The system may analyze current wait times, patient flow, patient volume, as well as presenting conditions of each patient to communicate estimated wait times to each patient.

In some embodiments, the virtual queues may process the estimated time for patients to be seen at the urgent care facility using the patient's current location. This estimated wait time is updated in real-time based on geo-fencing techniques and travel time to the urgent care facility.

In some embodiments, the system utilizes historical data received from the facility (e.g., seasonal increases in patients and/or certain treatments (such as injections during Flu season)) to educate estimated wait times. To perform this task, waiting room exit data may be utilized in tandem with geofencing data.

In some embodiments, the system utilizes machine learning algorithms to enhance the efficiency and accuracy of the app. The urgent care facility data is collected on a regular basis to train the machine learning models that contributes to continuous improvement in application's accuracy.

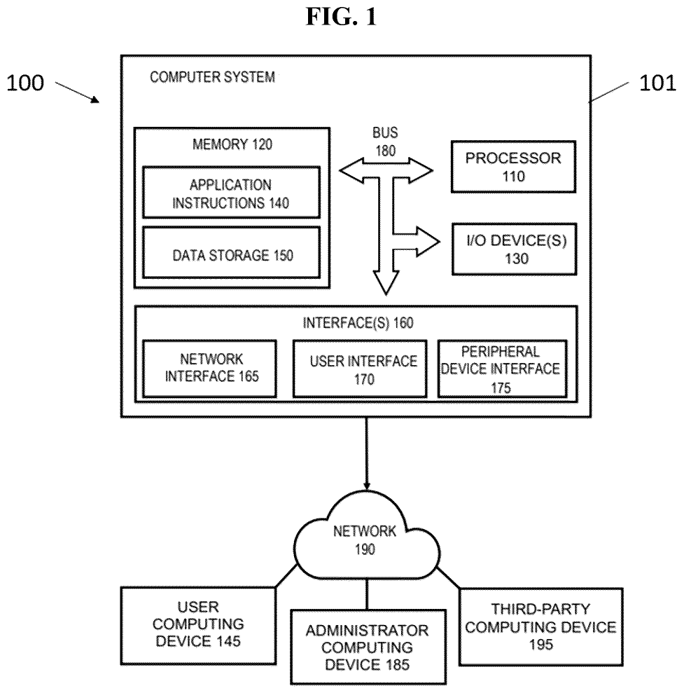

FIG. 1 illustrates an example of a computer system 101 that may be utilized to execute various procedures, including the processes described herein. The computer system 101 comprises a standalone computer or mobile computing device, a mainframe computer system, a workstation, a network computer, a desktop computer, a laptop, or the like. The computer system 101 can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

In some embodiments, the computer system 101 includes one or more processors 110 coupled to a memory 120 through a system bus 180 that couples various system components, such as an input/output (I/O) devices 130, to the processors 110. The bus 180 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

In some embodiments, the computer system 101 includes one or more input/output (I/O) devices 130, such as video device(s) (e.g., a camera), audio device(s), and display(s) are in operable communication with the computer system 101. In some embodiments, similar I/O devices 130 may be separate from the computer system 101 and may interact with one or more nodes of the computer system 101 through a wired or wireless connection, such as over a network interface.

Processors 110 suitable for the execution of computer readable program instructions include both general and special purpose microprocessors and any one or more processors of any digital computing device. For example, each processor 110 may be a single processing unit or a number of processing units and may include single or multiple computing units or multiple processing cores. The processor(s) 110 can be implemented as one or more microprocessors, microcomputers, microcontrollers, digital signal processors, central processing units, state machines, logic circuitries, and/or any devices that manipulate signals based on operational instructions. For example, the processor(s) 110 may be one or more hardware processors and/or logic circuits of any suitable type specifically programmed or configured to execute the algorithms and processes described herein. The processor(s) 110 can be configured to fetch and execute computer readable program instructions stored in the computer-readable media, which can program the processor(s) 110 to perform the functions described herein.

In this disclosure, the term “processor” can refer to substantially any computing processing unit or device, including single-core processors, single-processors with software multithreading execution capability, multi-core processors, multi-core processors with software multithreading execution capability, multi-core processors with hardware multithread technology, parallel platforms, and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures, such as molecular and quantum-dot based transistors, switches, and gates, to optimize space usage or enhance performance of user equipment. A processor can also be implemented as a combination of computing processing units.

In some embodiments, the memory 120 includes computer-readable application instructions 150, configured to implement certain embodiments described herein, and a database 150, comprising various data accessible by the application instructions 140. In some embodiments, the application instructions 140 include software elements corresponding to one or more of the various embodiments described herein. For example, application instructions 140 may be implemented in various embodiments using any desired programming language, scripting language, or combination of programming and/or scripting languages (e.g., C, C++, C#, JAVA, JAVASCRIPT, PERL, etc.).

In this disclosure, terms “store,” “storage,” “data store,” data storage,” “database,” and substantially any other information storage component relevant to operation and functionality of a component are utilized to refer to “memory components,” which are entities embodied in a “memory,” or components comprising a memory. Those skilled in the art would appreciate that the memory and/or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include, for example, RAM, which can act as external cache memory. The memory and/or memory components of the systems or computer-implemented methods can include the foregoing or other suitable types of memory.

Generally, a computing device will also include, or be operatively coupled to receive data from or transfer data to, or both, one or more mass data storage devices; however, a computing device need not have such devices. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, the steps and actions of the application instructions 140 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 140 for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 140 can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 140 can be downloaded to a computing/processing device from a computer readable storage medium, or to an external computer or external storage device via a network 190. A network adapter card or network interface in each computing/processing device receives computer readable program instructions from the network and forwards the computer readable application instructions 140 for storage in a computer readable storage medium within the respective computing/processing device.

In some embodiments, the computer system 101 includes one or more interfaces 160 that allow the computer system 101 to interact with other systems, devices, or computing environments. In some embodiments, the computer system 101 comprises a network interface 165 to communicate with a network 190. In some embodiments, the network interface 165 is configured to allow data to be exchanged between the computer system 101 and other devices attached to the network 190, such as other computer systems, or between nodes of the computer system 101. In various embodiments, the network interface 165 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol. Other interfaces include the user interface 170 and the peripheral device interface 175.

In some embodiments, the network 190 corresponds to a local area network (LAN), wide area network (WAN), the Internet, a direct peer-to-peer network (e.g., device to device Wi-Fi, Bluetooth, etc.), and/or an indirect peer-to-peer network (e.g., devices communicating through a server, router, or other network device). The network 190 can comprise copper transmission cables, optical transmission fibers, wireless transmission, routers, firewalls, switches, gateway computers and/or edge servers. The network 190 can represent a single network or multiple networks. In some embodiments, the network 190 used by the various devices of the computer system 101 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet). The Internet refers to the specific collection of networks and routers communicating using an Internet Protocol (“IP”) including higher level protocols, such as Transmission Control Protocol/Internet Protocol (“TCP/IP”) or the Uniform Datagram Packet/Internet Protocol (“UDP/IP”).

Any connection between the components of the system may be associated with a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer-readable media. In some embodiments, the computer-readable media includes volatile and nonvolatile memory and/or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer-readable storage media and/or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In some embodiments, the system can also be implemented in cloud computing environments. In this context, “cloud computing” refers to a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

As used herein, the term “add-on” (or “plug-in”) refers to computing instructions configured to extend the functionality of a computer program, where the add-on is developed specifically for the computer program. The term “add-on data” refers to data included with, generated by, or organized by an add-on. Computer programs can include computing instructions, or an application programming interface (API) configured for communication between the computer program and an add-on. For example, a computer program can be configured to look in a specific directory for add-ons developed for the specific computer program. To add an add-on to a computer program, for example, a user can download the add-on from a website and install the add-on in an appropriate directory on the user's computer.

In some embodiments, the computer system 101 may include a user computing device 145, an administrator computing device 185 and a third-party computing device 195 each in communication via the network 190. The user computing device 145 may be utilized to establish credentials, create a user profile, and otherwise interact with the features of the system. The third-party computing device 195 may be utilized by third parties to receive communications from the user computing device and/or administrative computing device 185.

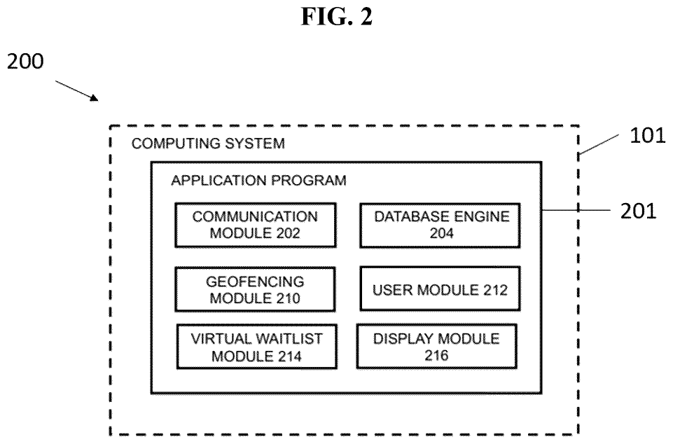

FIG. 2 shows an example computer architecture diagram 200 for the application program 201 operated via the computer system 101. Referring to FIG. 2, the computing system 101 operating the application program 201 comprises one or more modules having the necessary routines and data structures for performing specific tasks, and one or more engines configured to determine how the platform manages and manipulates data. In some embodiments, the application program 201 comprises one or more of a communication module 202, a database engine 204, a geofencing module 210, a user module 212, a virtual waitlist module 214, a display module 216.

In some embodiments, the communication module 202 is configured for receiving, processing, and transmitting a user command and/or one or more data streams. In such embodiments, the communication module 202 performs communication functions between various devices, including the user computing device 145, the administrator computing device 185, and a third-party computing device 195. In some embodiments, the communication module 202 is configured to allow one or more users of the system, including a third-party, to communicate with one another. In some embodiments, the communications module 202 is configured to maintain one or more communication sessions with one or more servers, the administrative/administrator computing device 185, and/or one or more third-party computing device(s) 195. In some embodiments, the communication module 202 allows each user to transmit and receive information which may be used by the system.

In some embodiments, a database engine 204 is configured to facilitate the storage, management, and retrieval of data to and from one or more storage mediums, such as the one or more internal databases described herein. In some embodiments, the database engine 204 is coupled to an external storage system. In some embodiments, the database engine 204 is configured to apply changes to one or more databases. In some embodiments, the database engine 204 comprises a search engine component for searching through thousands of data sources stored in different locations. The database engine 204 allows each user and module associated with the system to transmit and receive information stored in various databases.

In some embodiments, the database engine 204 derives various property data, mortgage data, tax data, insurance data, user data and the like such that the system may automatically generate a policy document based on user inputs.

In some embodiments, the geofencing module 210 allows the system to interpret the user's location and compare the user's location to the geofenced perimeter of a facility. This allows the system to determine if a patient has entered and/or exited a facility.

The computer system is coded to receive information input in various fields on the user interface. In such, the display module 216 provides each fillable field to the user and allows the user to input questions on the user interface. Information input by the user is transmitted, via the communication module 202 to the forms module 210 to allow for the automated generation of the policy document.

In some embodiments, the user module 212 facilitates the creation of a user account for the application system. The user module 212 may allow the user to input account information, establish user permissions and the like.

In some embodiments, the virtual waitlist module 214 is capable of analyzing a user's location, mode of transportation, and estimated time of arrival at the facility to then dynamically enter the user into a waitlist. The virtual waitlist module may then display the user's status in the waitlist to the user as well as facility administrators. Virtual waitlist module 214 can also provide dynamic waiting time estimates to patients, customers, and/or users. These estimates can be provided after one or more algorithms determine a user's location, use a total serving capacity of nearby UC(s), determine a total quantity of patients being served, determine a total quantity of patients already or currently waiting in a queue, and determining the user's number or estimated location in the queue.

In some embodiments, the display module 216 is configured to display one or more graphic user interfaces, including, e.g., one or more user interfaces, one or more consumer interfaces, one or more video presenter interfaces, etc. In some embodiments, the display module 216 is configured to temporarily generate and display various pieces of information in response to one or more commands or operations. The various pieces of information or data generated and displayed may be transiently generated and displayed, and the displayed content in the display module 216 may be refreshed and replaced with different content upon the receipt of different commands or operations in some embodiments. In such embodiments, the various pieces of information generated and displayed in a display module 216 may not be persistently stored. In some embodiments, display module 216 will receive commands such as to update the queue whenever there is a change in the conditions of the queue. These changes can occur when any user cancels his appointment. These changes can also occur based on an administrator from a UC changing or modifying the queue. These changes can also occur based on a patient being served or detained, late, tardy, or otherwise not on time for their appointment.

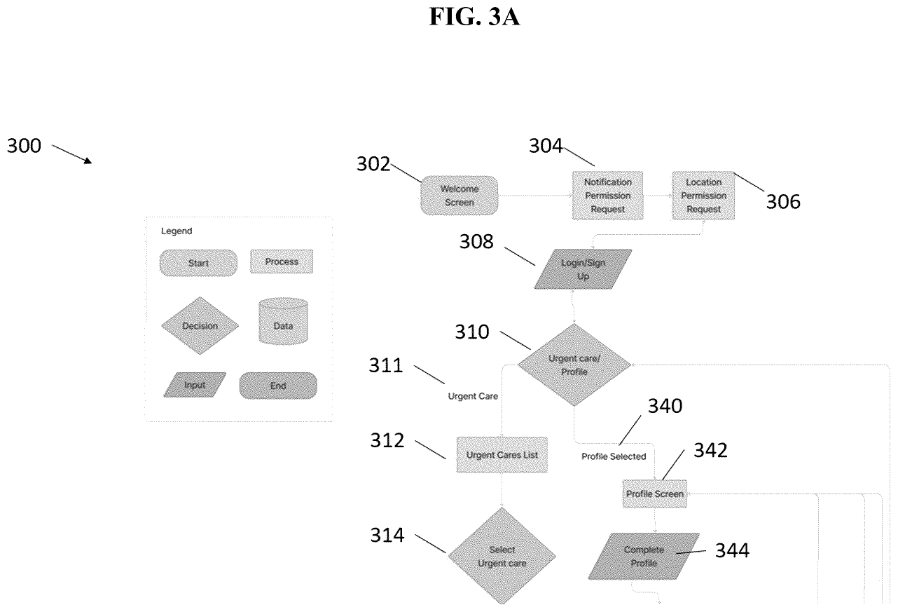

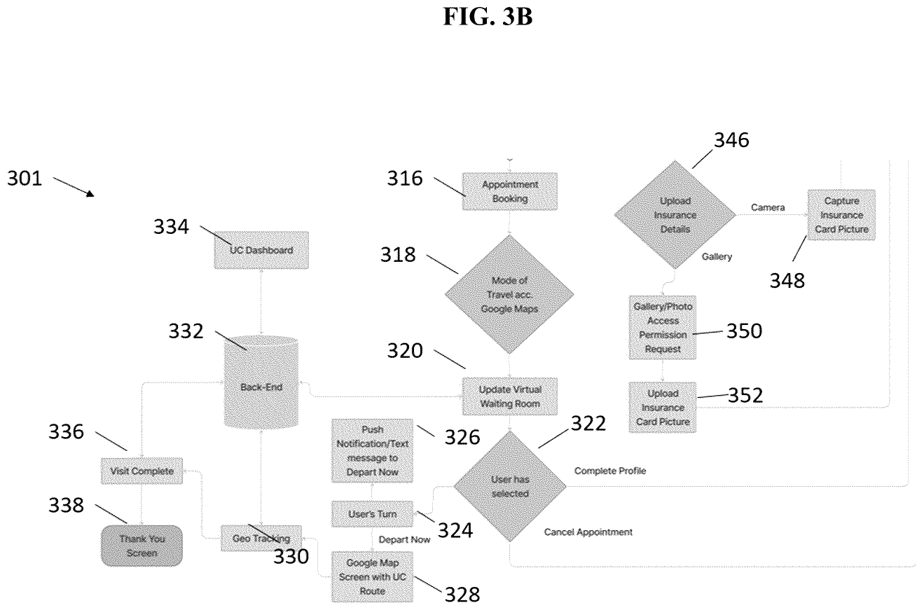

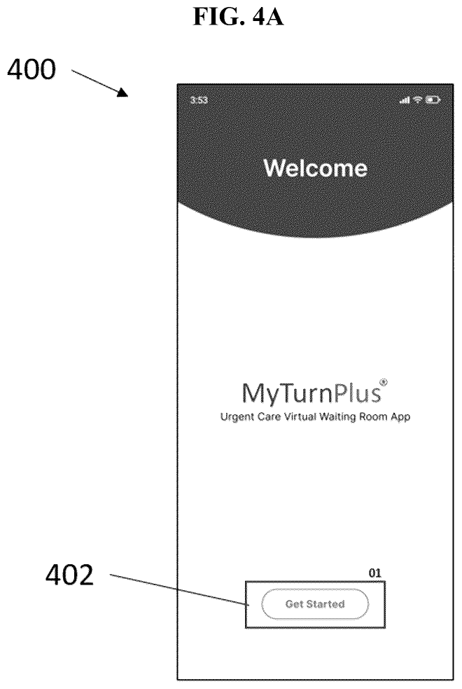

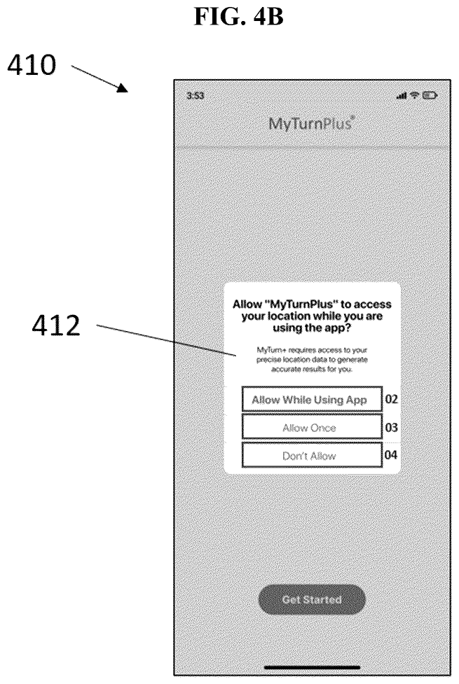

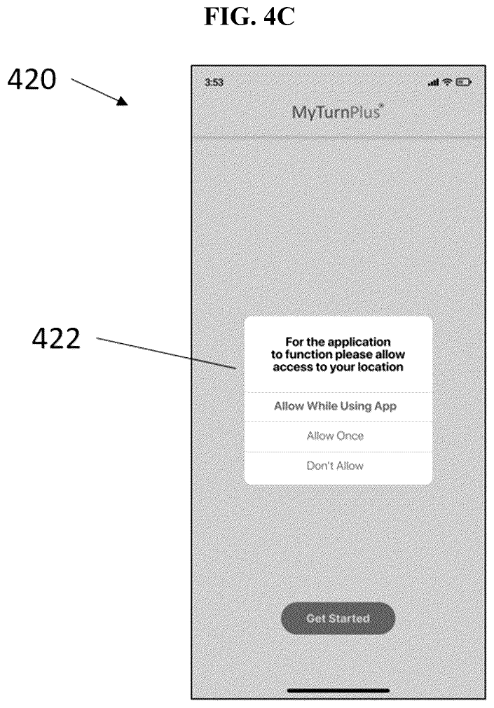

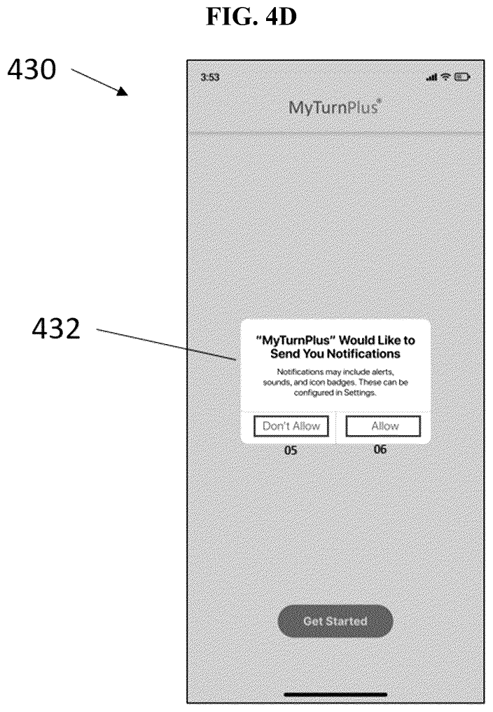

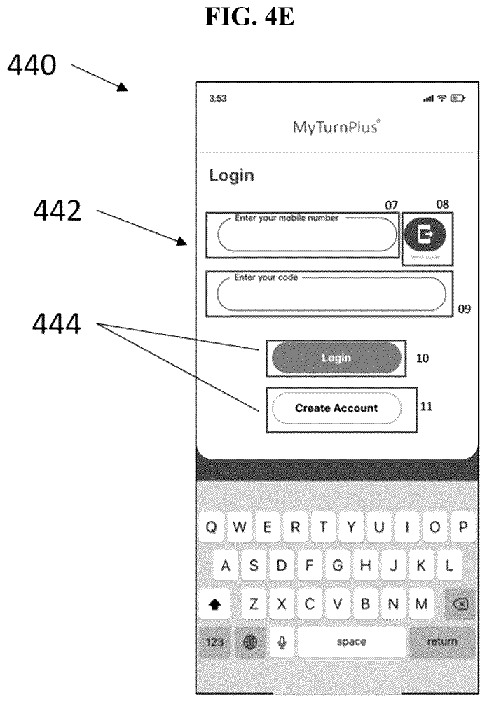

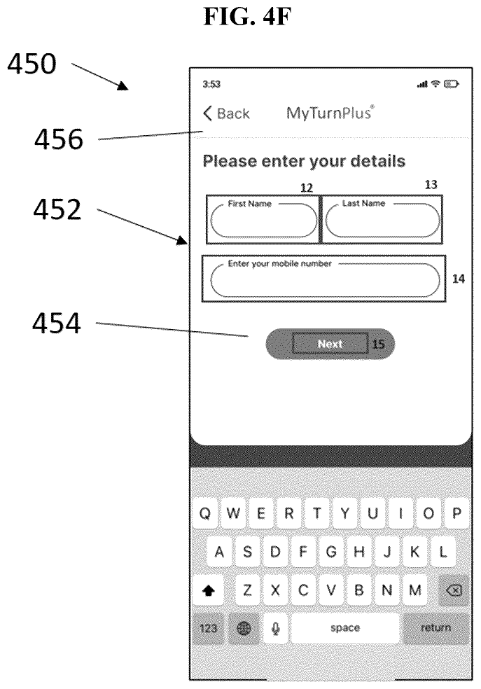

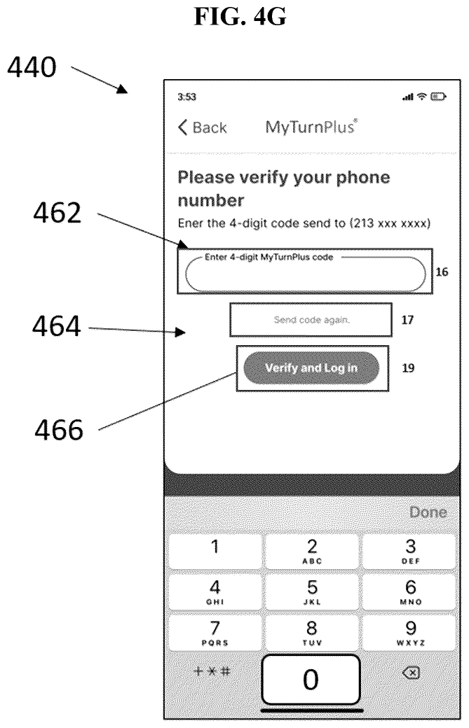

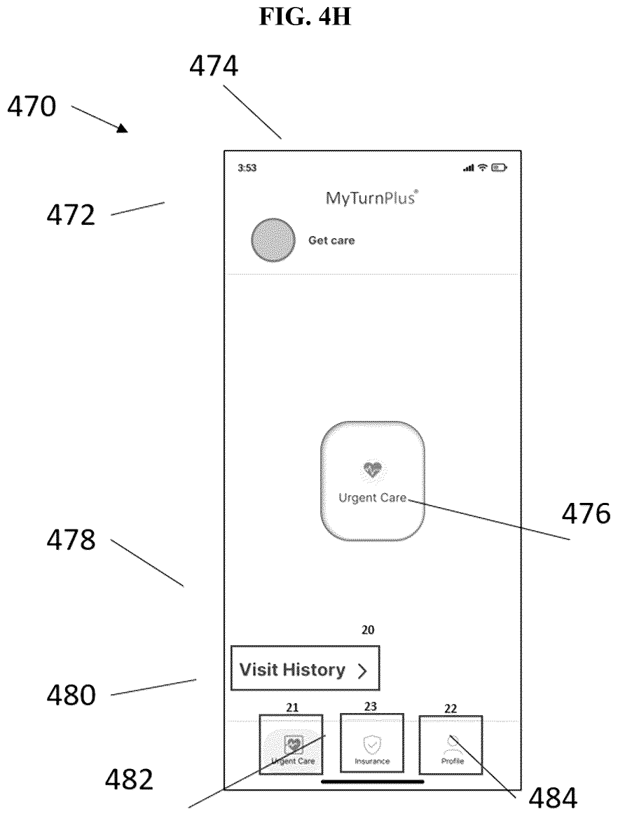

FIGS. 3A-3B illustrates a block diagram 300, 301 of an application flow which the system may provide and a user may interact with while utilizing the system, according to an example embodiment. Initially, users can download a mobile application to their mobile phone (which may be a smartphone, tablet, or the like). The system may cause the phone to display a welcome screen 302 when the user accesses the application on their mobile phone (see FIG. 4A and associated description for additional detail). A notification permission request may be displayed to the user at step 304, which the user can accept or decline by selecting the corresponding option (see FIG. 4D and associated description for additional detail). Next, a location permission request can be displayed to the user at step 306, which the user can accept or decline by selecting the corresponding option (see FIGS. 4B-4C and associated descriptions for additional detail). The location permission request acceptance can allow for various operations of the application to utilize location services to determine the current location of the mobile phone and thus, the user. A login/sign up screen can be displayed in step 308 that allows a user to login and/or register for the system with their mobile phone number, email address, screenname, or otherwise by entering the appropriate information into corresponding fields (see FIG. 4F and associated description for additional detail). The mobile number can be verified with a one-time password (OTP) that the user must use to log in, or through other means (see FIG. 4E, 4G and associated descriptions for additional detail). The registered mobile number can then be used to log into the application in the future, along with a password that the user selects, biometric information, personal identification number (PIN), or other verification.

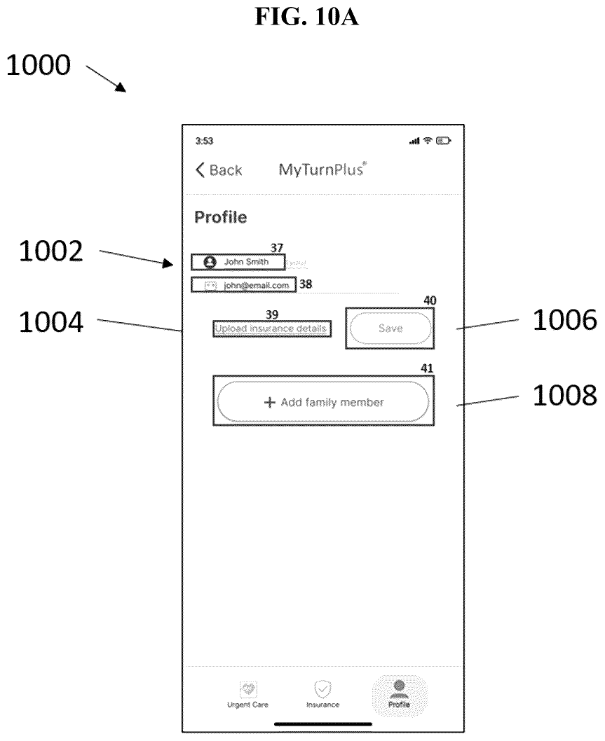

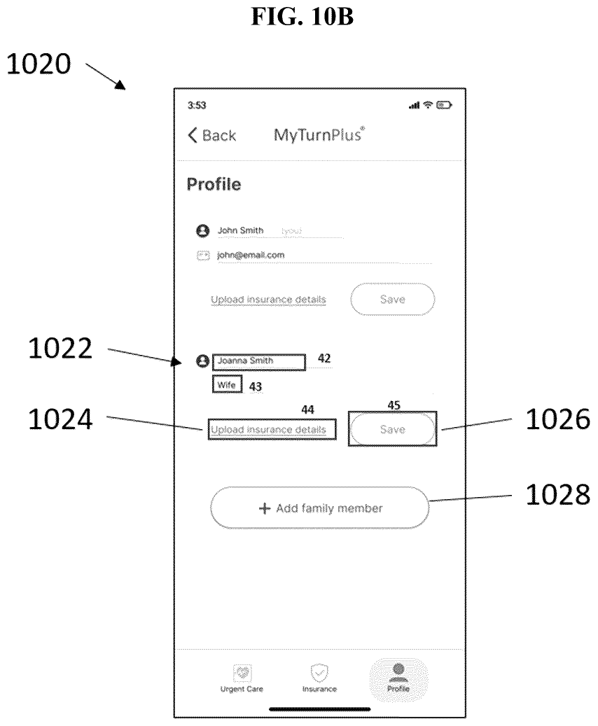

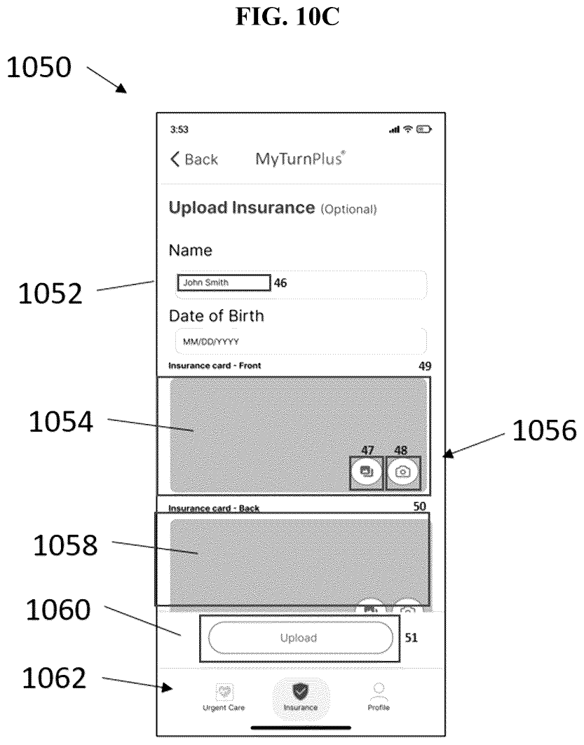

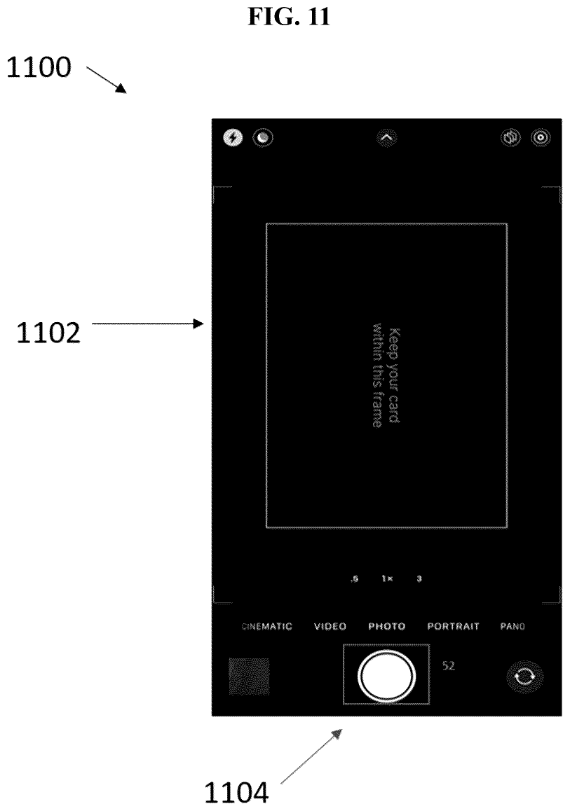

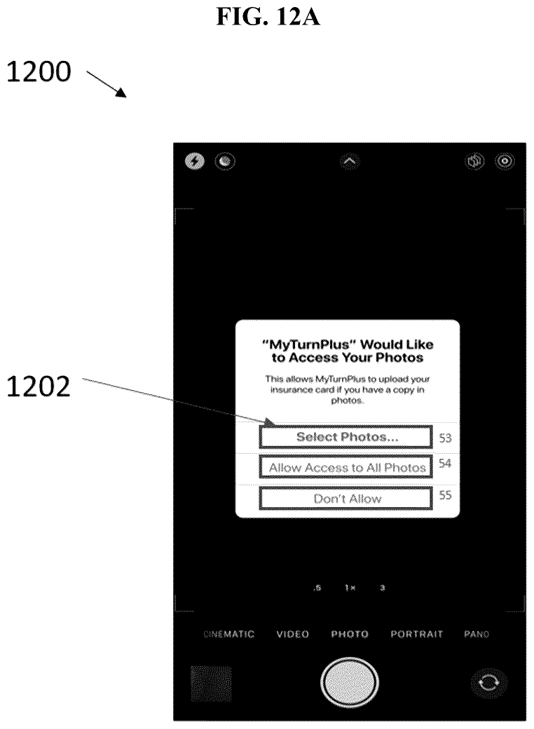

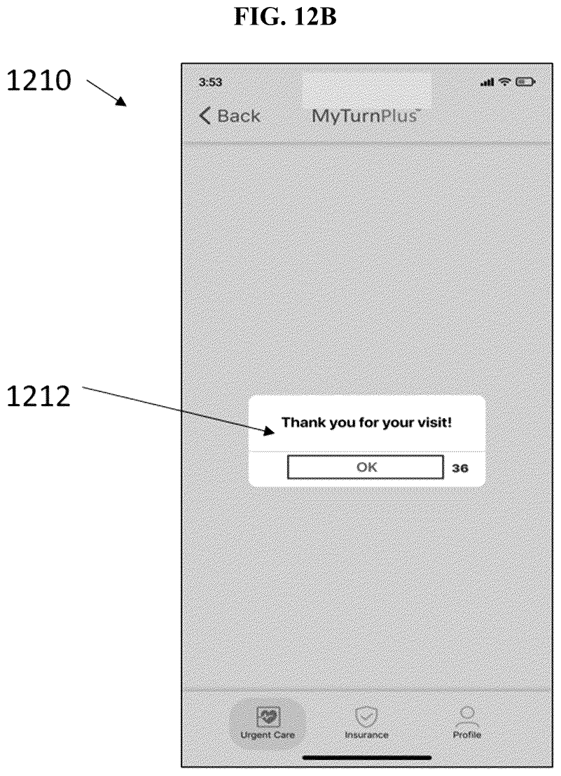

If the user is a new user, the user may access a user profile with personal data in step 310. Otherwise, if the user is a previously registered user, the user can access their profile in step 310 (see FIG. 10A and associated description for additional detail). In some embodiments, step 310 can also display urgent care information. In some embodiments, the user also has the option to add profile of family members, upload the family members' health insurance cards, and complete any required verification (see FIG. 10B and associated description for additional detail). Users can select a profile among multiple connected profiles (e.g. for a family) in step 340. Step 342 is a profile display screen whereby the system displays a profile screen or screens with pertinent profile data and/or information. If a profile has yet to be completed, the user can perform any required completion in step 344. In step 346, a user can upload insurance details or information, such as uploading or entering information for a personal health insurance card, and the system can verify the information (see FIG. 10C and associated description for additional detail). A communicatively coupled camera of the wireless device can be used to capture insurance information, such as by taking an insurance card picture, scanning a Quick Response (QR) code or other code, or otherwise in step 348 (see FIG. 11 and associated description for additional detail). In step 350 a user can also elect to access stored information such as captured images in their wireless phone or device's memory in response to a gallery/photo access permission request (see FIG. 12A and associated description for additional detail). After making the selection, the user can upload the insurance card picture to the system in step 352. After steps 348 and/or 352, the system can return to the profile screen 342. In some embodiments, the system can store these insurance card images, e.g. in an AWS S3 bucket, whereafter this picture may only be accessed via a system server which is HIPAA Compliant and deployed on, e.g. EC2 (AWS). This server may provide insurance card image information (and/or related) only to authorized administrator user(s) in compliance with any applicable legal requirements.

Insurance cards and/or documents and/or data contained therein can be automatically verified in some instances, for example, through the system linking with insurance carrier systems. In various embodiments insurance information may alternatively or additionally be verified by administrators manually or through other processes.

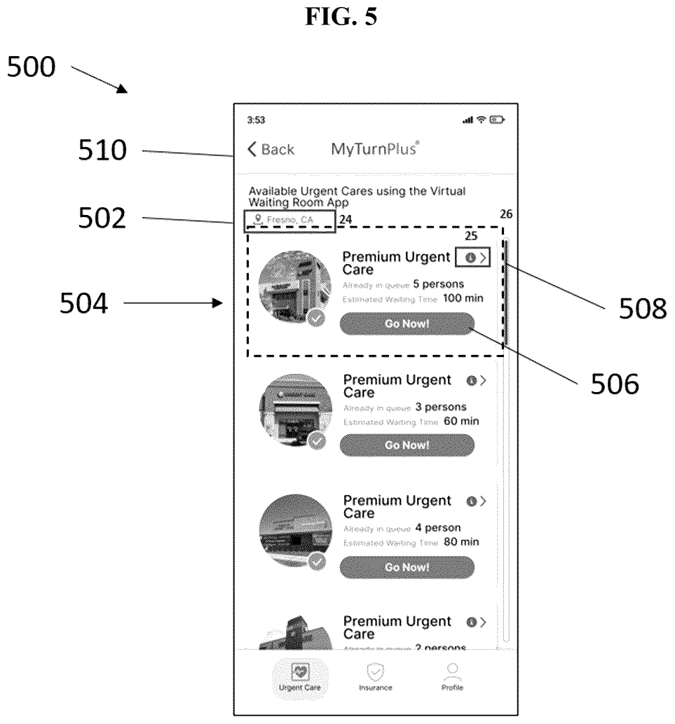

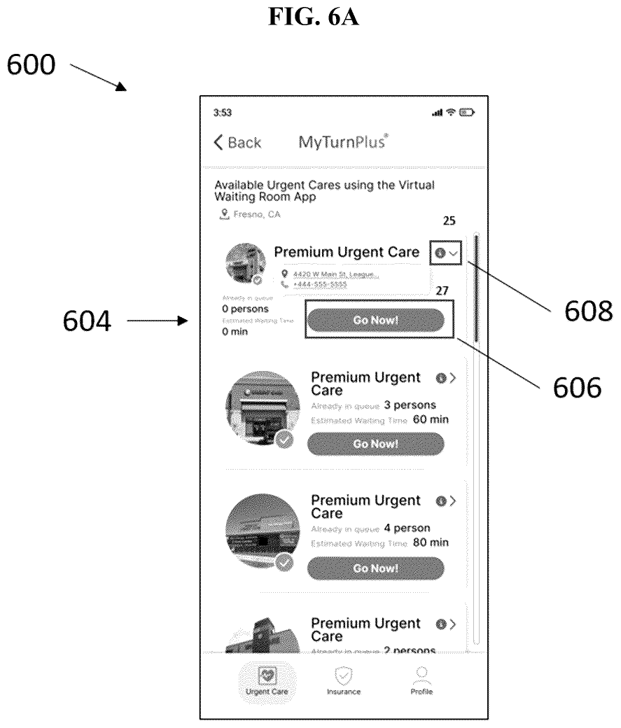

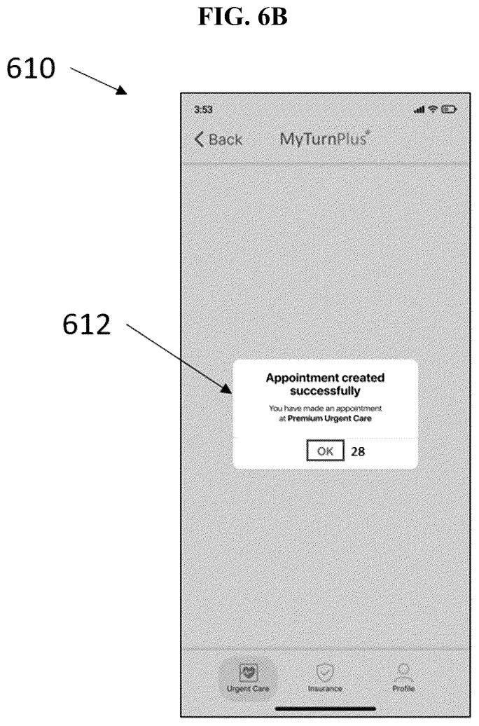

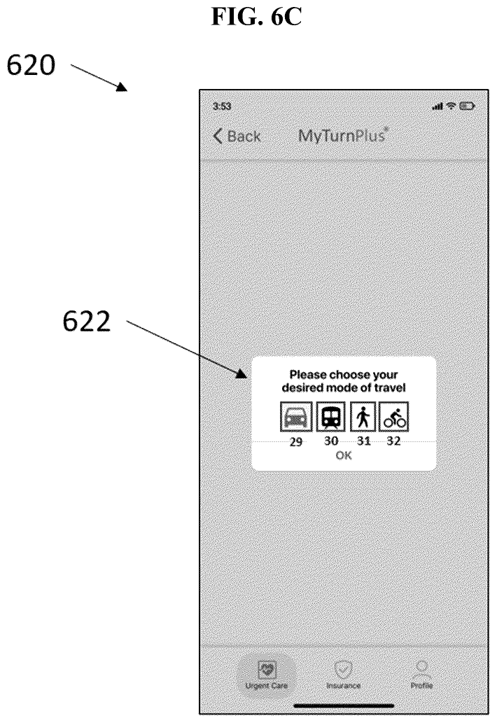

In step 310, a user may elect to view urgent care(s). Upon selecting an urgent care button in step 311, the system can, based on the current user location, provide a list of recommended nearest, fastest, and/or most convenient urgent care centers to the user for selection by the user in step 312 (see FIG. 5 and associated description for additional detail). In some embodiments a maximum distance may be set by a system administrator in order to ensure quality (e.g. 5 mile maximum distance from the user's current or selected location). The user then selects an urgent care in step 314. Next the user can book an appointment in step 316 by setting and/or selecting appointment information (e.g. time, date, or others) (see FIG. 4A and associated description for additional detail). Upon appointment confirmation (see FIG. 6C and associated description for additional detail), in step 318 the system/application asks the user to select a mode of travel (e.g., car, transit, bike, walk) from the user's current location to the selected urgent care facility (see FIG. 6B and associated description for additional detail). The travel mode information can be linked to a third-party service or application (e.g. Google™ Maps, which may provide or contribute to calculating of an estimated time of when the user will arrive at the selected urgent care facility for treatment. Travel mode can also contribute to algorithmic calculation of an estimated time a user requires to see a doctor at an appointment time for scheduling and/or for updating the queue time purposes for the user and/or other users. The system can take into account such information and in step 320, update a virtual waiting room for the selected urgent care facility.

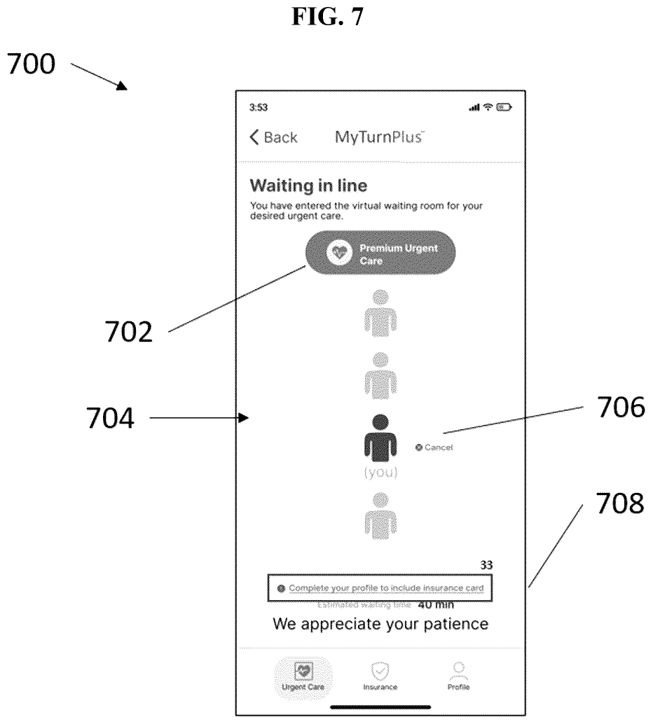

A virtual waiting room can include a virtual queue display in the application program (see FIG. 7 and associated description for additional detail). The virtual queue can show a total number of persons in the queue along with user's standing (i.e., their current place in the queue or line). In step 322, the user can then select an option to view profile and be taken to profile screen 342. Alternatively, the user could cancel the appointment. Alternatively, the system may tell the user when their turn at the urgent care facility will be occurring in step 324. If a user elects to cancel their appointment, a waiting time in the queue for other users will be reduced by system servers automatically according to one or more appropriate algorithms.

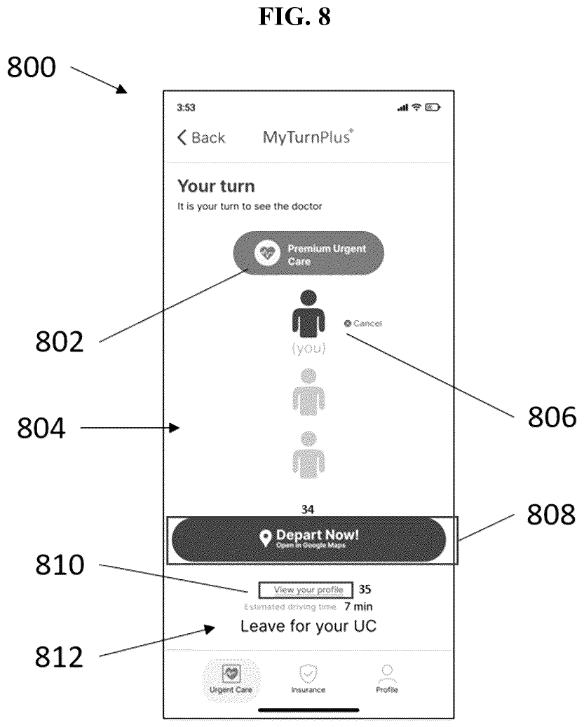

As a result of step 324 and if the user has elected to, the system can provide one or more push notification(s), text message(s), or other notification(s) to the user to depart their current location in order to attend their appointment at the appropriate time in step 326 (see FIG. 8 and associated description for additional detail). This can be a “WHEN TO LEAVE” message so the user knows when to leave their current location (e.g. residence) as determined by and/or according to the location of the user. This can help the user to avoid unwanted waiting at urgent care center and help them to arrive at or at least with minimum waiting time at the urgent care facility. This can also help the urgent care facility prevent spread of disease or illnesses by reducing the number of people who are physically present at a location in a waiting room at a given time. As such, it operates as a “just in time” or minimum waiting time process.

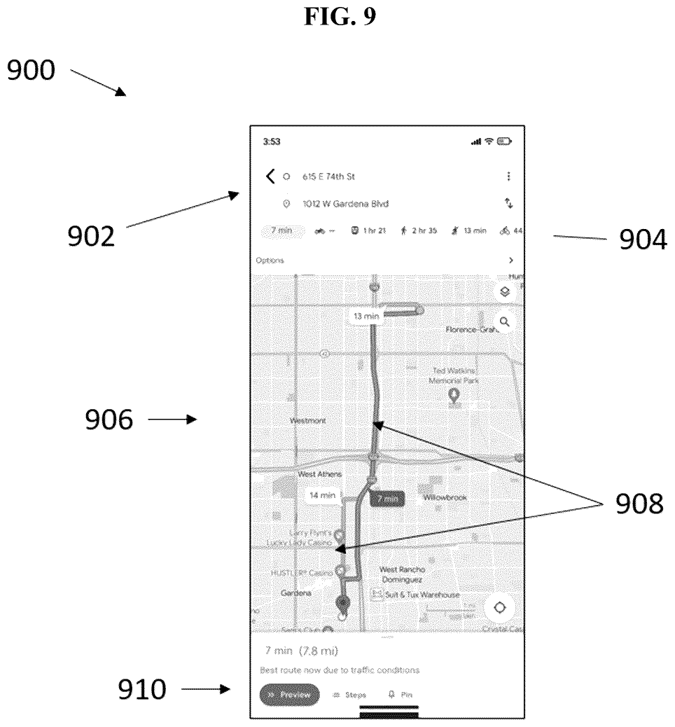

A third-party location and/or map application (e.g. Google™ Maps) can be displayed with one or more routes to the selected urgent care facility in step 328 (see FIG. 9 and associated description for additional detail). In general, a shortest navigation route may be used, while others can include avoiding particular obstacles (e.g. toll roads, etc.). When notified by the application program to leave for the urgent care facility, the user may receive the option to show directions in such a map interface. The system may employ geo-tracking and/or geofencing techniques in step 330 that is coupled to back-end 332 and that tracks the entrance to and/or departure from an area by a user from the set parameters of the selected urgent care facility. This can also update the virtual queue in the front-end application based these parameters. Back-end 332 can also update a linked virtual waiting room 320. Likewise, back-end 332 can be coupled with an urgent care dashboard 334. Similarly, back-end 332 can track when a visit is complete in step 336, which may also be coupled with geo-tracking 330. Upon completion of a visit, the system application may display a thank you screen 338 (see FIG. 12B and associated description for additional detail).

FIGS. 4-12 provide mobile device user interface screens for UC patient users.

FIG. 4A shows a splash screen 400 of a mobile device user interface, according to an example embodiment. As shown in the example embodiment, a get started button 402 can allow the user to start an appointment scheduling process and/or login/registration when selected by a patient user.

FIG. 4B shows a location permissions screen 410 of a mobile device user interface, according to an example embodiment. As shown in the example embodiment, a popup 412 can include one or more buttons requesting a user to set location permissions. These can include allow location only while using the application, allow once, don't allow, or others. Allow location only while using the application can prevent or cause the popup not to be displayed again and allow location services while using the application. Allow once can allow a single use of location services, whereafter the popup may appear during a next session. Don't allow can prevent the use of location services and may inhibit use of other features of the application. Generally, if a user elects not to allow their location to be tracked and/or used, the system application may prompt the user to allow the location on one or more subsequent occasions. Otherwise, the system may restrict the user's usage of one or more capabilities of the system.

FIG. 4C shows a location permissions follow-up screen 420 of a mobile device user interface, according to an example embodiment. As shown in the example embodiment, a popup 422 can be displayed if the user chooses not to allow permission for location services, for example by selecting don't allow on a location permission screen (see FIG. 4B and associated description). In order for aspects of the application to properly function the application again requests location permission, via popup 422 which can include one or more buttons requesting a user to set location permissions. These can include allow location only while using the application, allow once, don't allow, or others. Allow location only while using the application can prevent or cause the popup not to be displayed again and allow location services while using the application. Allow once can allow a single use of location services, whereafter the popup may appear during a next session. Don't allow can prevent the use of location services and may inhibit use of other features of the application.

FIG. 4D shows a notification permissions follow-up screen 430 of a mobile device user interface, according to an example embodiment. As shown in the example embodiment, a popup 432 can be displayed which includes buttons that allow the user to allow or not allow notifications to be sent and/or displayed by the user's mobile device. If permitted, users can receive permissions in the form of alerts, sounds, icon badges, flashing camera lights and/or screens, and/or others. If denied, the application will not provide notifications for users.

FIG. 4E shows a login screen 440 of a mobile device user interface, according to an example embodiment. As shown in the example embodiment, once a UC facility scheduling application implementing the methods and systems described herein has been downloaded and installed on a user mobile device, the user can interact with various buttons and enter personal information in fields 442. These can include a phone number field, send code field, enter code field, login field, and create account field, among others. Depending on whether a user has already registered or is creating an account, they can select the appropriate button 444 from login or create account. If a registered user has entered their phone number the user can send a one-time password (OTP) that they will receive in the form of a short message service (SMS) or text message on their registered mobile phone device. Once received, the user can enter the received OTP in the OTP field, select a login field to log into the UC scheduling application or otherwise. If a user has forgotten their password, they can select a recovery account field that will take them to other display screens that provide the user access by validating their identity in a different manner (e.g. answering a secret question, providing an account number, or otherwise).

FIG. 4F shows an account creation screen 450 of a mobile device user interface, according to an example embodiment. As shown in the example embodiment, a user can enter personal details such as their name, mobile number, or others in appropriate corresponding fields 452. Then the user can select a next button 454 which will take them to a next screen with a further step for account creation. A back button 456 can cause the system application to display a previous screen to the user.

FIG. 4G shows verify contact number screen 460 of a mobile device user interface, according to an example embodiment. As shown in the example embodiment, if the user has received an OTP the user can enter the number in OTP confirmation field 462 and select verify and log in button 466, which will cause the system to check the validity of the OTP and grant or deny access based on such validity. If the user did not receive the OTP, they can select a button 464 that will send a new OTP to the user, for instance by SMS text, email, or otherwise.

FIG. 4H shows an on-boarding page display screen 470 of a mobile device user interface, according to an example embodiment. As shown in the example embodiment, a home button 474 can display a home screen when selected by a user. A get care display 472 may be a graphic and/or text that shows a user's identifying information, such as a first initial and last name. An urgent care button 476 can display a list of urgent care facilities to a user when selected. A visit history button 478 can cause the system to display a list of previous visits and/or cancellations when selected. In some embodiments this list can be interactive, such that a user can select one or more of the visits/cancellations in the list to view additional information. An urgent care button 480 can display local urgent cares, according to geographic location information based on the location of the UC facilities and the user mobile device. An insurance button 482 can display insurance information associated with the user who is signed into the application or otherwise has a currently active profile that is being used. A profile button 484 can display profile information regarding the user who is signed into the application.

FIG. 5 shows an urgent care facility listing screen 500 of a mobile device user interface, according to an example embodiment. As shown in the example embodiment, location information 502 can be selectable by a user and/or automatically updated according to a location of the mobile device. In some embodiments, this is a selectable button and a user can change the location. Urgent care facility information 504 can include at least one graphic or image associated with a UC facility, a name of the UC facility, a number/quantity of persons in the UC facility's queue, an estimated waiting time, and/or other information. Also shown is a “Go Now!” or other selectable button 506 whereby a user can select the button and set the associated UC facility as a destination. In some embodiments, the system can automatically hide UC locations (even if they may be near a user location) if the UC is operational hours are set to close or expire within 30 minutes or less (or as set or modified by an administrator or otherwise, e.g. minutes estimated waiting time and/or travel time will exceed that of the appointment available at the UC). Additional information button 508 can cause the system to display additional information about the associated UC facility when selected by a user, as shown in FIG. 6. A back button 510 can cause the system to display a previous screen when selected by a user.

FIG. 6A shows an urgent care facility listing screen 600 of a mobile device user interface with expanded information, according to an example embodiment. As shown in the example embodiment, if a user selects an additional information button 608, the system can display additional information associated with the UC facility. This can include an address or other location information; a phone number, email, or other contact information; an updated number of persons located in a queue, an updated waiting time, and/or other information. A Go Now! Button 606 can cause the system to begin an appointment setting process.

FIG. 6B shows a travel mode selection screen 610 of a mobile device user interface with expanded information 612, according to an example embodiment. As shown in the example embodiment, a user can select at least one travel mode for at least one portion of a journey to a UC facility. Here, automobile, bus, walk, and bicycle options are shown, but others are possible as well. Estimated travel times can be calculated and displayed by the system prior to and/or upon selection of a travel mode.

FIG. 6C shows a successful appointment creation screen 620 of a mobile device user interface with expanded information, according to an example embodiment. As shown in the example embodiment, upon successful creation of an appointment the system can display a popup 622 or other message confirming the appointment has been successful. A user confirmation button can allow the user to clear the message when selected.

FIG. 7 shows an urgent care facility queue screen 700 of a mobile device user interface, according to an example embodiment. Pertinent information displayed can include, for example, that the registered user is waiting in the queue and they have entered the virtual waiting room for their desired urgent care. As shown in the example embodiment, selected UC facility information 702 can include a name and/or other information. A real-time queue display 704 can include indicators representing the number of people currently waiting in the queue for the selected UC facility. A user indicator 706 can indicate the user's location in the queue display 704, for example by showing the user as an avatar with a different color than others in the queue. The user indicator 706 can also include one or more buttons that perform various actions, such as a cancel button, which may cancel the user's appointment and remove them from the queue. System message 708 can display a user interactive button in some embodiments, such as notifying the user that they should complete their profile with insurance card(s) as soon as possible. Selecting message 708 may display the appropriate screen for the user to interact with. In some embodiments, if a user moves or otherwise navigates away from urgent care facility queue screen 700 and then attempts to book one or more appointments at one or more other UC locations, the user will be prevented from accomplishing such additional booking. Instead, the user will be prompted that they are already in a queue and the system will navigate back to screen 700.

FIG. 8 shows an updated urgent care facility queue screen 800 of a mobile device user interface, according to an example embodiment. Pertinent information displayed can include, for example, that it is currently or soon to be the user's turn to see the doctor. As shown in the example embodiment, selected UC facility information 802 can include a name and/or other information. A queue display 804 can include indicators representing the number of people currently waiting in the queue for the selected UC facility. A user indicator 806 can indicate the user's location in the queue display 804, for example by showing the user as an avatar with a different color than others in the queue. As shown, the user indicator 806 is first in the queue display 804, indicating that the user is next to see the doctor. A depart now button 808 can be user selectable in some embodiments, and upon selection by the user may display a map interface showing the user a route from their current location to the selected UC facility (e.g. see FIG. 9 and associated description). The depart now button 808 can be displayed by the system or otherwise become visible on an urgent care facility queue screen of a mobile device when the user's travel time is equal to their waiting time, as calculated by the system using one or more integrated mapping screens. As such, this can be accomplished by comparing the estimated travel time between a user's current location and the time their appointment is scheduled to begin. Other calculations and metrics can also be included in various embodiments including buffer time, which may allow for potential travel delays and/or early or delayed completion of prior scheduled appointments by other users. A view your profile button 810 can display a user profile screen to the user if selected by the user. The system application can also display messages 812 such as a real-time calculation of an estimated travel time to the selected UC facility and/or information about when the user should leave their current location based on the selected mode(s) of transport to make their appointment at the scheduled time.

FIG. 9 shows a map screen 900 of a mobile device user interface, according to an example embodiment. In various embodiments, the map screen displayed can be integrated from a third-party interface (e.g. using a Google® Map application) screen. This allows for the system to include navigation information from a location of the mobile device that the user is using to the selected UC after the user selects a depart now button (e.g. 808 of FIG. 8) or a link sent via short messaging service (SMS) message, email, or otherwise to the user. As shown in the example embodiment, a current location and UC facility location can be shown as address(es) 902 and may be interactive or modifiable in some instances. Travel modes 904 can allow a user to modify how they plan to travel to the selected UC facility. For example, a user may change their travel mode from bicycle to automobile or walking, and this may cause the system to update the route, including the estimated time and directions. Map 906 can include location indicators, roads, train tracks, parks, blocks, points of interest, and/or other pertinent map details. One or more routes 908 can be displayed on map 906 and may allow users to select between alternative routes. A currently selected route may be indicated, for example with a different color than alternate routes. Map buttons 910 can be interactive and can include steps—which may be turn-by-turn directions, preview—which may be an overview of the selected route, pin—which may add a pin to the map, and/or others. In some embodiments, in the event that the system application session is terminated (e.g. the user closes the application on the mobile device) or the user is not otherwise using the application, the user may receive one or more push notifications from the system and/or a SMS message, email, or other communication that can include a map link and a message that it is currently time for the user travel to the scheduled UC so the user will not miss their appointment.

FIG. 10A shows a user profile editing screen 1000 of a mobile device user interface, according to an example embodiment. As shown in the example embodiment, interactive user name and contact information fields 1002 can be provided and allow a user to set and/or modify the corresponding information. Upload insurance details button 1004 allows a user to input or upload insurance information and can take a user to an insurance upload screen such as the one described and shown with respect to FIG. 10C. A save button 1006 allows the user to save any progress and/or edits. An add family member button 1008 allows a user to add family members or others they are closely associated with and may share insurance information with, such as the family member edit screen shown and described with respect to FIG. 10B.

FIG. 10B shows a family member profile editing screen 1020 of a mobile device user interface, according to an example embodiment. As shown in the example embodiment, interactive family member name and contact information fields 1022 can be provided and allow a user to set and/or modify the corresponding information. Upload insurance details button 1024 allows a user to input or upload insurance information for the family member and can take a user to an insurance upload screen such as the one described and shown with respect to FIG. 10C. A save button 1026 allows the user to save any progress and/or edits. An add family member button 1028 allows a user to add additional family members or others they are closely associated with and may share insurance information with.

FIG. 10C shows an insurance upload screen 1050 of a mobile device user interface, according to an example embodiment. As shown in the example embodiment, a user can enter and/or modify their name in a username field 1052, for example by entering information via user interface buttons on a touchscreen display. An insurance card front image 1054 can be displayed for user review and/or reference. Users can select one or more buttons 1056 that can allow them to access images saved in memory for upload (see FIG. 12 and associated description for additional detail) or to capture an image via a camera (see FIG. 11 and associated description for additional detail) of the wireless device for upload. An insurance card back image 1058 can also be displayed for user review and/or reference. An upload button 1060 can allow the user to upload the information to the system when selected by the user. Navigation buttons 1062 can allow a user to select an urgent care, select insurance, select a profile, and/or others and can cause the system to display the associated screen for the user to view and interact with.

FIG. 11 shows a camera screen 1100 of a mobile device user interface, according to an example embodiment. In various embodiments a user can aim a camera of the mobile device at an insurance card or other document with insurance information and the device will display the image to be captured in a display window 1102. The user can then capture the image by selecting the capture button 1104. Then, the device can store the image in memory and/or the user can upload it to the system.

FIG. 12A shows an image selection screen 1200 of a mobile device user interface, according to an example embodiment. As shown in the example embodiment, if a user has saved an image of an insurance card or document in a camera roll or other memory of a mobile device, the system can prompt the user to grant access to images for the system application. A system prompt 1202 can include one or more user interactive buttons such as select photos, allow access to all photos, don't allow access, or others.

FIG. 12B shows a thank you screen 1210 of a mobile device user interface, according to an example embodiment. As shown in the example embodiment, a thank you popup 1212 or other message can be displayed by the system when a user has completed their UC facility visit, e.g. as when they depart a geofenced location at the UC facility where they attended an appointment. Selecting an ok button can close the prompt and/or application.

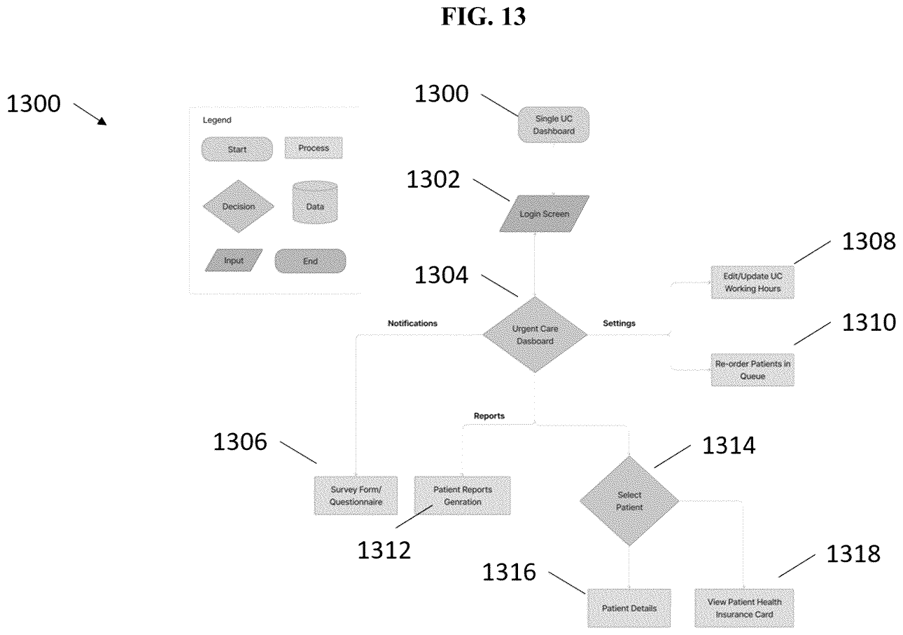

FIG. 13 shows a block diagram 1300 of an administrative flow, according to an example embodiment. This flow allows administrative users to view waitlist status of each patient scheduled to visit the facility, as well as perform other administrative functions. As shown, a single UC dashboard 1302 can be accessed by a user by providing credentials at a login screen 1302. Next, an urgent care dashboard 1304 can provide various functions, such as providing notifications for a survey form/questionnaire 1306, allowing setting adjustments such as edit/update UC working hours 1308 and re-ordering patients in a queue 1310, and generating reports. In some embodiments, upon login, users can view and edit information according to their role. One or more backend system servers can provide restriction to accessing various forms of information and functions based on a user's logging into a profile with a particular role. Roles may be updated by administrator users.

Reports can include patient reports generation 1312 and individual reports by selecting a patient 1314 and then creating reports including patient details 1316 and view patient health insurance card 1318.

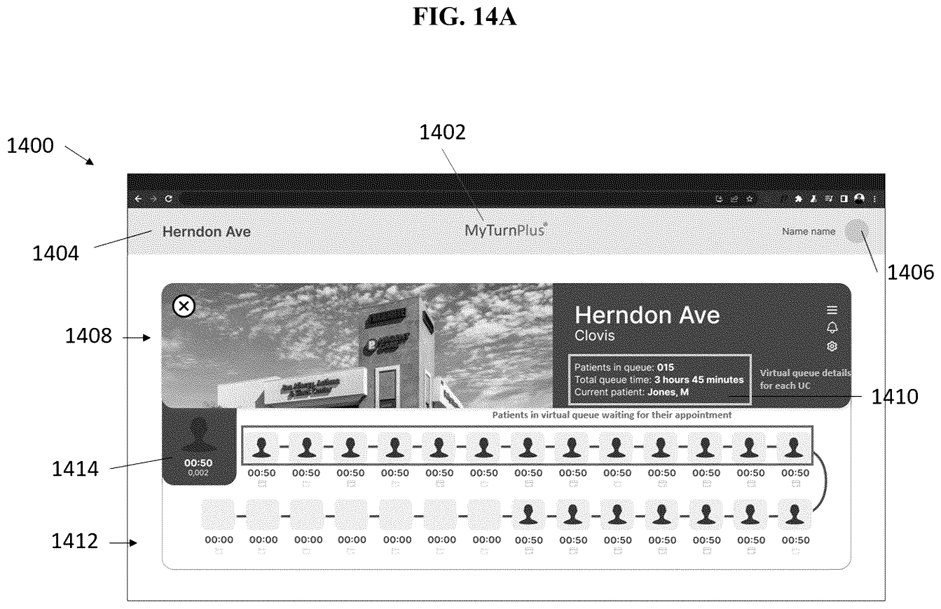

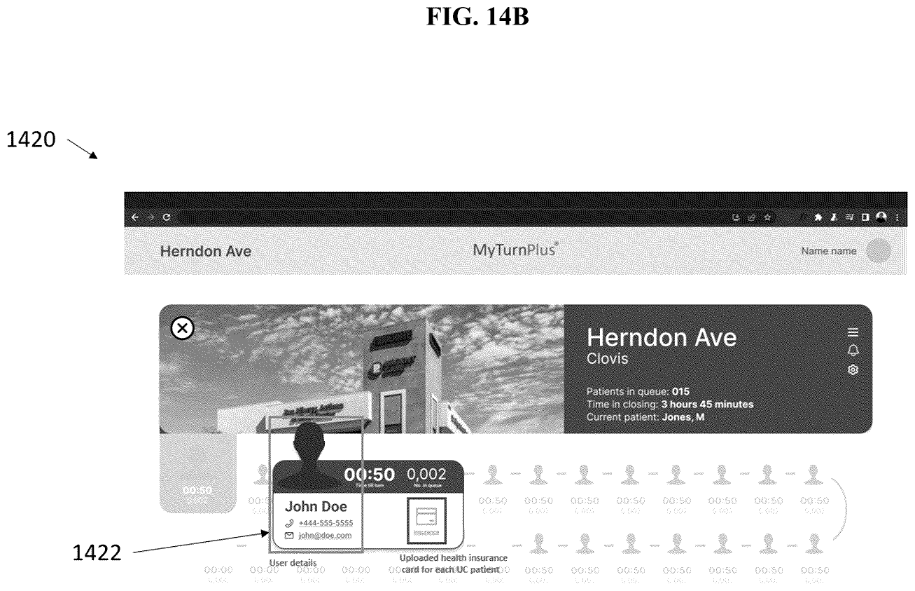

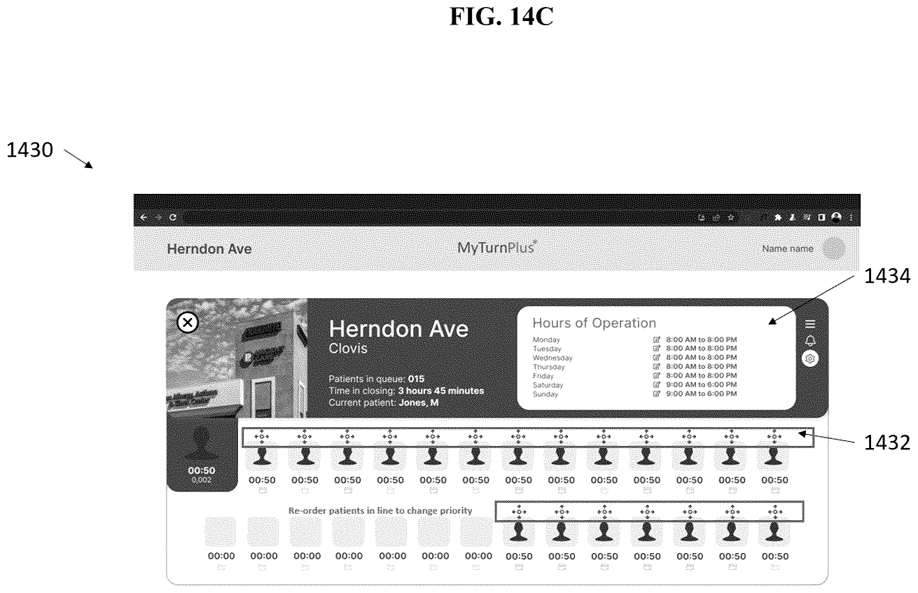

FIGS. 14-16 illustrate exemplary screenshots of the various user interfaces provided to the administrative user.

FIG. 14A shows an urgent care facility queue information administrator user interface screen 1400, according to an example embodiment. As shown in the example embodiment, a home button 1402 can cause the system to display a home screen. An administrator screen can include the name of a currently displayed UC facility 1404 that can change when another facility is selected. User profile information 1406 can include an avatar or other image for the user and a username and/or real name of a user who is logged into the system. UC facility information 1408 can include one or more images associated with the currently selected UC facility, the UC facility name and location data, menu button, alert button, settings button, exit button, and/or others. Virtual queue details 1410 for the selected UC facility queue can include a number of patients in the UC facility's queue, total queue time, current patient name, and/or others. Virtual queue 1412 can include avatars for each patient and indicators or the ability to select each patient to view information such as name, reason for visit, insurance information, past visits, expected appointment time/duration, and/or others. A current patient 1414 can have their information stand out slightly from the queue by setting them slightly apart from the queue and in a different color.

FIG. 14B shows an urgent care facility queue information administrator user interface screen 1420, according to an example embodiment. As shown in the example embodiment, if an administrator user selects a patient user 1422 in the queue they can view personal details such as name and contact information and select uploaded insurance information for viewing and/or verification.

FIG. 14C shows an urgent care facility queue information administrator user interface screen 1420, according to an example embodiment. Administrator user interface screen 1420 can allow an administrator user to change, modify, and/or update appointments of users using the system. As shown in the example embodiment, if an administrator user selects patient users in the queue, they can reorder them by selecting their avatar 1432 and moving it in order to change priority and/or location in the queue. If the administrator represents a single UC facility that is not part of a larger group, they can edit operating hours by day, opening and closing times, and other information 1434.

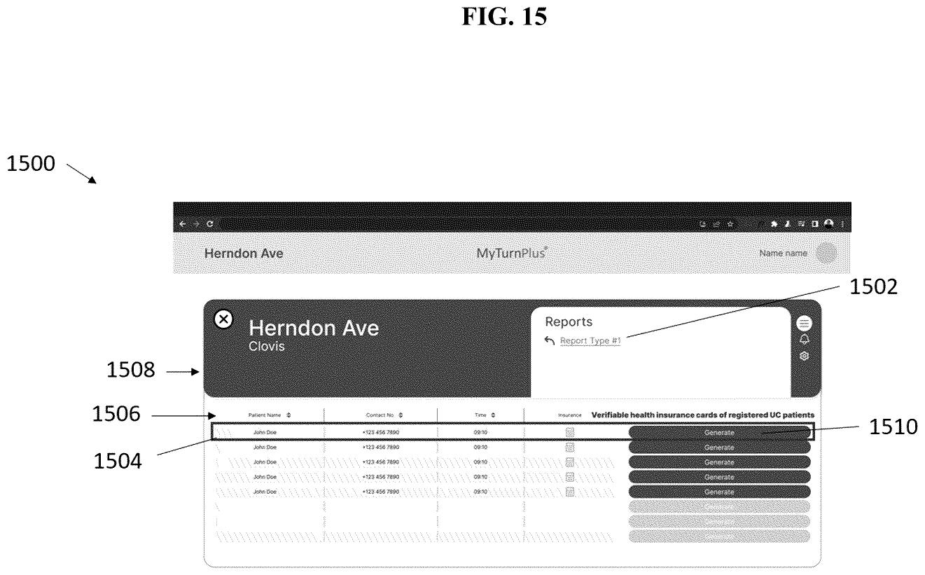

FIG. 15 shows an urgent care facility report administrator user interface screen 1500, according to an example embodiment. As shown in the example embodiment, when a menu button has been selected by a user, the user can choose one or more reports 1502 to view. Once selected, the user can view data in the report. In the example embodiment, a list 1506 of UC facility patients using the system application can be displayed, with information such as patient names, contact information, times of appointments, insurance information, and others. For an individual UC patient 1504, an administrator user can also select a report generation button 1510 that will generate a report about the individual user. Also shown is UC facility information 1508 that can include one or more images associated with the currently selected UC facility, the UC facility name and location data, menu button, alert button, settings button, exit button, and/or others.

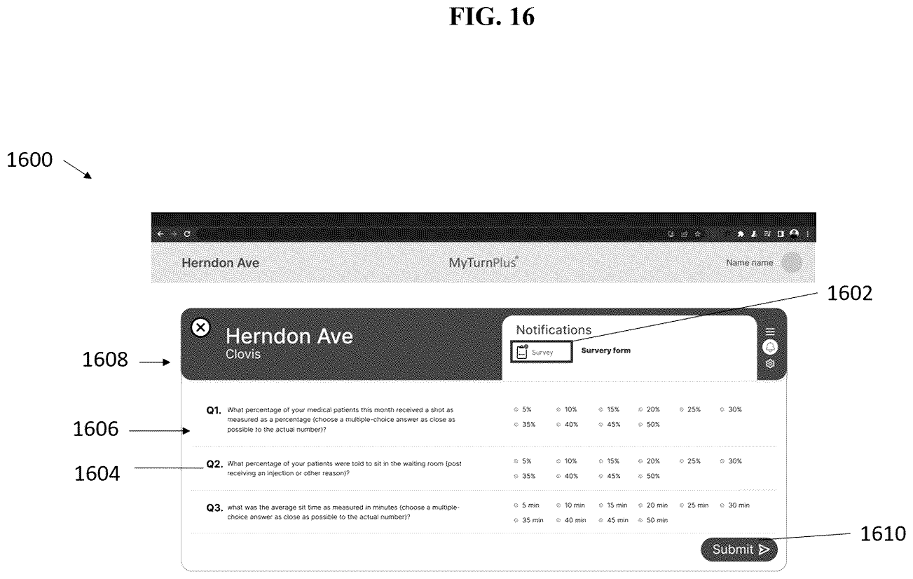

FIG. 16 shows an urgent care facility notification administrator user interface screen 1600, according to an example embodiment. As shown in the example embodiment, when an alert button has been selected by a user, the user can choose one or more notifications 1602 to view. In the example embodiment the notification 1602 shown is a survey form button. Once selected, the user can view questions in the survey. In the example embodiment, a list 1606 of UC facility survey questions can be displayed, with optional answer choice buttons and/or fields included. For an individual survey question 1604, an administrator user can, for example, select a radio button to choose their answer to the survey question. Once complete, the administrator user can select a submit button 1610. Also shown is UC facility information 1608 that can include one or more images associated with the currently selected UC facility, the UC facility name and location data, menu button, alert button, settings button, exit button, and/or others.

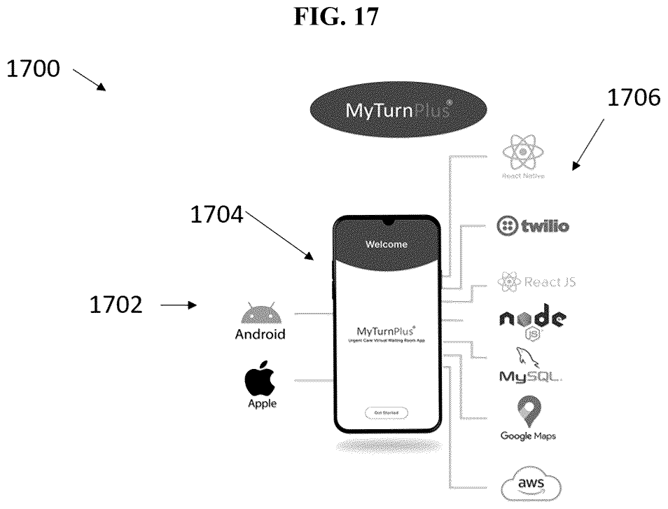

FIG. 17 shows system integration diagram 1700, according to an example embodiment. As shown in the example embodiment, webstores such as the Apple™ store or Android™ store can allow a user to download the system application to their mobile device 1704. Integration with a number of third-party services can also be provided, such as React Native™, Twilio™, React JS™ Node Js™, MySQL™, Google Maps™, AWS™, and/or many others. These example services can provide functionality as follows: React Native™: an open-source JavaScript framework, designed for developing android and iOS mobile applications. The application can use React Native™ as its front-end for the design and development of android and iOS™ applications. React JS™: an open-source JavaScript framework, designed for developing web applications. The application can use React JS™ as its front-end for the design and development of admin portals for the system application. Laravel™: an open-source PHP framework used for backend development. The MTP application will use Laravel as its back-end framework that will connect with React Native™ for mobile applications and with React JS™ for web applications. MySQL™: It is an open-source relational database management system. Node.js® open-source, cross-platform JavaScript runtime environment. The data for the system application can be stored in MySQL database. Google Maps™: The system application can use Google Maps™ API to enable directions for its users. The system user gets direction from the residence to booked UC via Google Maps™. AWS™: Amazon Web Services offers reliable and scalable cloud computing services. The system application can be hosted on AWS™. In some embodiments, AWS EC2 can be used for serving deployment and cloud computation of algorithms; AWS RDS can be used for hosting MySQL database; AWS security groups can be used to restrict access of all users to access the resources; and AWS S3 can be used to store images/docs like assets and HIPAA Compliant. Nginx can be used for reversed proxy. Zero SSL can be used for securing a system backend.

In some embodiments, surveys can be automatically generated by a super administrator dashboard. The system can force data to be submitted by a particular day of the month or a red flag may be engaged by the system for the UC facility. This red flag may inform an administrator user that the system requires the survey to be completed in order to maintain the most accurate and up to date information. Surveys can lay the foundation for a strong database that allows the system to provide advanced machine learning functionality.

In this disclosure, the various embodiments are described with reference to the flowchart illustrations and/or block diagrams of methods, apparatus (systems), and computer program products. Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc. that perform particular tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” “module,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a user a list of options), scroll bars (which allow a user to move up and down a window) and buttons (which can be “pushed” with a click of a mouse). A wide variety of applications is known to those in the art.

The phrases “Application Program Interface” and API as are used herein mean a set of commands, functions and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer's job easier, and it also benefits the end user since it generally ensures that all programs using the same API will have a similar user interface.

The phrase “central processing unit” as is used herein means a computer hardware component that executes individual commands of a computer software program. It reads program instructions from a main or secondary memory, and then executes the instructions one at a time until the program ends. During execution, the program may display information to an output device such as a monitor.

The term “execute” as is used herein in connection with a computer, console, server system or the like means to run, use, operate or carry out an instruction, code, software, program and/or the like.

It should be understood that the methods, systems, platforms, and processes described herein cannot be performed as mental exercises solely in the human mind and are not abstract ideas.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

What is claimed is:

1. A method for providing a real-time virtual medical facility queue on a mobile device via a network, comprising:
instructions stored in memory of the mobile device that, when executed by a processor of the mobile device, cause the device to perform operations including:
displaying, on a display of the mobile device, data corresponding to at least one local medical facility based on a location of the mobile device;
displaying, on a display of the mobile device, a virtual queue of patients pending treatment at the medical facility; and
updating the virtual queue based on geographic movement of the mobile device.

2. The method of claim 1, wherein the instructions, when executed by a processor of the mobile device, further cause the device to perform operations including:
scheduling an appointment based on user selection of an appointment confirmation button displayed on the mobile device.

3. The method of claim 2, wherein the instructions, when executed by a processor of the mobile device, further cause the device to perform operations including:
providing a mobile device notification indicating a departure time based on the location of the mobile device.

4. The method of claim 3, wherein the instructions, when executed by a processor of the mobile device, further cause the device to perform operations including:
updating the departure time based on geographic movement of the mobile device.

5. The method of claim 1, wherein the instructions, when executed by a processor of the mobile device, further cause the device to perform operations including:
transmitting insurance data for a user of the mobile device to the medical facility via the network.

6. The method of claim 5, wherein the insurance data is captured using a camera of the mobile device.

7. The method of claim 1, wherein the instructions, when executed by a processor of the mobile device, further cause the device to perform operations including:
updating the virtual queue based on user selection of a mode of travel.

8. The method of claim 1, wherein the instructions, when executed by a processor of the mobile device, further cause the device to perform operations including:
offering, on a display of the mobile device, a list of local medical facilities for user selection.

9. The method of claim 1, wherein updating the virtual queue based on geographic movement of the mobile device further comprises:
determining a location of the mobile device with respect to the medical facility; and
removing a user indicator associated with the mobile device from the virtual queue when the mobile device travels outside of a preset proximity distance from the medical facility.

10. The method of claim 9, wherein the present proximity distance from the medical facility is monitored via geofencing.

11. An application server for providing a real-time virtual queue for a medical facility via a network, comprising:
at least one processor of the server executing instructions stored in memory that cause the server to perform operations including:
sending virtual queue data for the medical facility to a user mobile device via the network for display on the user mobile device.

12. The application server of claim 11, wherein the medical facility is selected from a list of medical facilities by a user of the user mobile device.

13. The application server of claim 12, wherein the list of medical facilities is populated according to a geographic location of the user mobile device.

14. The application server of claim 11, wherein executing the instructions causes the server to perform operations further comprising:
sending updated virtual queue data based on a geographic location change of the user mobile device.

15. The application server of claim 11, wherein executing the instructions causes the server to perform operations further comprising:
sending updated virtual queue data based on a geographic location change of a third-party user mobile device.

16. The application server of claim 11, executing the instructions causes the server to perform operations further comprising:
sending updated virtual queue data based on a cancellation received from a third-party mobile device via the network.

17. The application server of claim 11, executing the instructions causes the server to perform operations further comprising:
storing insurance data received from the user mobile device.

18. The application server of claim 17, wherein the insurance data was captured using a camera of the user mobile device.

19. The application server of claim 11, executing the instructions causes the server to perform operations further comprising:
sending updated virtual queue data based on selection of a mode of travel on the user mobile device.

20. The application server of claim 11, executing the instructions causes the server to perform operations further comprising:
updating virtual queue data based on an appointment cancellation received from the user mobile device.
